(19) United States
(12) Patent Application Publication
(10) Pub. No.: US 2019/0247237 A9
(43) Pub. Date: Aug. 15, 2019

(54) Protective Film For Application to An Individual's Body

(71) Applicants: Monica Ladd-Kimbrough, Minden, NV (US); Mark Jones, Gardnerville, NV (US); Beth Hickey, Arlington, TX (US)

(72) Inventors: Monica Ladd-Kimbrough, Minden, NV (US); Mark Jones, Gardnerville, NV (US); Beth Hickey, Arlington, TX (US)

(21) Appl. No.: 15/299,902

(22) Filed: Oct. 21, 2016

Related U.S. Application Data
(60) Provisional application No. 62/366,451, filed on Jul. 25, 2016.
(60) Provisional application No. 62/359,409, filed on Jul. 7, 2016.
(60) Provisional application No. 62/244,358, filed on Oct. 21, 2015.

(51) Int. Cl. A61F 13/02; A61F 13/10; A61M 35/00; A61F 13/00; A45D 29/00; A45D 31/00

(52) U.S. Cl. CPC A61F 13/0253; A61F 13/105; A61M 35/00; A61F 13/0266; A61F 2013/00272; A45D 29/007; A45D 31/00; A45D 29/001; A61F 13/00063

(57) ABSTRACT

According to an aspect of the disclosure, a protective film for application to a portion of an individual's body, a method for applying a protective film to a portion of an individual's body, and an apparatus with a protective film for application to a portion of the individual's body are provided.

### CROSS REFERENCE TO RELATED APPLICATIONS

The present disclosure claims benefit and priority to U.S. Provisional Patent Application Ser. No. 62/244,358 entitled “An Artificial Nail Apparatus Configured to Deliver a Health Additive to a Fingernail”, filed on Oct. 21, 2015, U.S. Provisional Patent Application Ser. No. 62/302,913 entitled “Protective Nail Film”, filed on Mar. 3, 2016, U.S. Provisional Patent Application Ser. No. 62/359,409 entitled “Protective Nail and Skin Film”, filed on Jul. 7, 2016, and U.S. Provisional Patent Application Ser. No. 62/366,451 entitled “Protective Nail and Skin Film”, filed on Jul. 25, 2016, which are each hereby incorporated by reference in their entirely as though fully set forth herein.

### TECHNICAL FIELD OF THE DISCLOSURE

The present disclosure generally relates to a protective film for application to a portion of an individual's body and in particular, to the individual's nail or skin. The protective film may include one or more health additives incorporated therein and being configured to enhance the condition of the nail or skin by repairing or minimizing the damage caused by, and/or protecting the nail or skin from, external elements or products. The protective film may also be configured to be a releasing mechanism for the removal of products, such as decorative treatments, from the nail. Further, the protective film may be incorporated into an apparatus for application onto a portion of the individual's body.

### BACKGROUND OF THE DISCLOSURE

Daily contact with products having artificial ingredients can cause damage to an individual's body over time. This is particularly true when it comes to the application, long time wear, and removal of nail polish on an individual's finger or toe nails.

Nail polishes dale back centuries and have been used as a way for people to decorate their nails. These polishes generally consist of a lacquer or gel that can be applied to a user's finger or toe nails. These polishes are known to come in different colors so that an individual can change the color of their nails, such as to coordinate with an outfit or the like. To provide durability from cracking, chipping, etc., these polishes often include additives or other chemicals to make them harder and more durable. Certain health concerns have been attributable to the use of nail polishes, particularly if the use is extensive.

The process of removing nail polish from a user's nails is also known to create potential damage to a user's health as well as the nails themselves. In particular, damage can be caused to the various layers of the nail. As shown in FIGS. 1A and 1B, the layers of the nail 10 may include, but are not limited to, the dorsal (upper layer) 12, intermedial (middle layer) 14, and the ventral (lower layer) 16. Typically, nail polish is removed with nail pads or nail polish removers. Nail polish removers typically consist of an organic solvent, but may also include oils, scents, and/or coloring. Nail polish remover packages are also known. One type of remover includes individual felt pads soaked in remover that can be rubbed against the nail to remove the polish. Another type of remover is known to consist of a bottle of liquid remover that can fee applied to a cotton ball or cotton pad, which in turn can be applied to the nail to remove the polish. Still another type of remover can consist of a container filled with foam that allows a use to insert a finger into the container to engage the foam and then twist the finger until the polish comes off. Choosing a type of remover is often determined by the user's preference and often the price or quality of the remover. The most common solvent used in these removers is an acetone, which is a powerful and effective chemical that can be harsh on skin and nails.

Given the known issues with applying and removing nail polish from an individual's nails, other ways of decorating nails have been developed including gel polishes and artificial or acrylic nails.

Gel polishes are very popular as they are known to last a long time on the nail plate and have increased durability and improved performance over conventional nail polishes. To apply gel polishes, the nail plate is first buffed lightly then the gel is applied and cured with an Ultra Violet or LED light. Additionally, these gel polishes continue to cure with any UV light or LED light including the sun outside, which further increases its durability. This makes the gel harder and the bond to the underlying nail plate tighter. Gel polishes are also known to create difficulty with respect to the removal process. The process for removing the gel generally may consist of soaking the nail and attached gel in a solvent which is made up of acetone for a period of time. Another process can include wrapping and securing cotton balls or pads soaked in acetone around the finger and fingernail for a period of time. After approximately thirty minutes of soaking, a metal scraper is generally used to dig, peel, pry, or scrape the gel off. Because the gel has bonded to the nail, 2 to 3 of the seven layers (as shown in FIGS. 1A and 1B) of the nail plate are often subsequently removed, which can lead to severe nail damage and brittleness over time.

Similarly, artificial fingernails are known in the art for overlying natural fingernails to provide for a desired aesthetic affect. Conventional artificial fingernails are typically made of an opaque and hard plastic. These nails are generally attached to the natural fingernails with an adhesive and can be detached from the natural fingernails by later removing the adhesive. After the artificial nails are applied, they are typically coated with a nail polish and/or a finishing material. Alternatively, after the artificial nails are applied, acrylic may be applied over the entire nail with nail polish or finishing material being applied afterwards. The nail polish can be used to make the artificial nails look like the natural nails, although it can also be used to provide other attractive and unique artificial nail appearances. The process for the removal of artificial or acrylic nail may be the same as the process for the removal of gel polish.

An issue with conventional artificial or acrylic nails is that they may inhibit the healthy development of the underlying natural fingernail and may even allow for the growth of bacteria or other harmful organisms between the natural fingernail and the artificial or acrylic nail. Accordingly, there remains a need for an improved artificial or acrylic nail.

It would thus be desirable to provide a system or method for decorating nails that improves on prior decorative nail processes as well as enhances the natural nail, protects the natural nail from lacquer polishes, gel polishes, and acrylics, protects the natural during removal of the same, and acts as a releasing mechanism for the same as well as provides an improved artificial nail.

In addition to nail polishes and similar treatment doing damage to the nail, the same may do damage to the surrounding skin. Further, other products, chemicals, external elements such as friction caused by clothing or shoes, or environmental elements may also cause damage to the skin. Resulting in the skin to crack or for calluses, blisters, or the like to form. For instance, an individual may develop a callus on their foot because of the shoes they wear.

As such, it would thus be desirable to provide a system or method that not only protects and repairs the nail from decorative nail processes, but also, repairs the surrounding skin or other portion of the user's skin that have cracks, calluses, blisters, and the like.

### SUMMARY OF THE DISCLOSURE

It is an aspect of the present disclosure to provide a protective film for application to an individual's body. Another aspect of the present disclosure may include a method for applying a protective film to an individual's body. An apparatus with a protective film for application to the individual's body is also provided.

It is a further aspect of the present disclosure to provide a protective film for decorating nails that improves on prior decorative nail processes as well as enhances the natural nail, protects the natural nail from lacquer polishes, gel polishes, artificial and/or acrylic nails, protects the natural during removal of the same, and acts as a releasing mechanism for the same.

According to one aspect of the present disclosure, a system and method are for decorating nails that utilize a membrane film over an individual's nail such that a decorative treatment can be applied overtop of the membrane film are provided.

It is an aspect of the present disclosure that the system for decorating nails includes a membrane film having an adhesive for applying the film over the individual's nails.

It is a further aspect of the present disclosure that the adhesive on the membrane film includes health additives or actives for repairing, protecting, and strengthening the individual's nail. The health additives or actives may be time-released actives that are dispersed to the nail after a predetermined amount of time after application of the film. In an alternative aspect, the health additives or actives may be non-time released actives that are dispersed to the nail immediately when the film is applied to the nail.

It is another aspect of the present disclosure to provide a system and method for protective film that is designed to repair damaged skin surrounding the nail or apart from the nail that has experience damage, utilizing a membrane film over that portion of the individual's skin.

It is another aspect of the protective film that the membrane film has an adhesive for applying the film to the skin, which contains health additives or actives for repairing, protecting, and strengthening the skin. The health additives or actives may be time-released actives that are dispersed to the skin after a predetermined amount of time after application of the film. In an alternative aspect, the health additives or actives may be non-time released actives that are dispersed to the skin immediately when the film is applied to the skin.

According to another aspect of the disclosure, an apparatus is provided. The apparatus includes a nail body having an upper surface and a lower surface. A protective film with an adhesive is disposed against the lower surface for securing the nail body to a natural fingernail of a person. The adhesive may be separated into a plurality of beads that are spaced from one another by a plurality of gaps. At least one health additive is disposed against the lower surface of the nail body in the gaps for being delivered onto the natural fingernail after the lower surface of the nail body has been applied against the natural fingernail. Accordingly, an aspect of the present disclosure provides for an apparatus that is not only aesthetically pleasing, but is configured to release at least one health additive that encourages healthy development of the natural fingernail and inhibits the growth of bacteria or other harmful organisms between the apparatus and the natural fingernail.

According to a further aspect of the disclosure, the grid shaped pattern of the adhesive and health additive on the lower surface of the nail can provide for an even distribution of the adhesive and health additive. The even distribution of the adhesive and health additive ensures that the apparatus remains secured to the fingernail while also ensuring an even distribution of the health additive onto the natural fingernail.

According to yet another aspect of the disclosure, the apparatus is easy to position on a fingernail. More specifically, prior to positioning the artificial nail on the natural fingernail, a backing membrane may easily be removed from the nail body, revealing the protective film with an adhesive, allowing the individual lo place the nail body on their fingernail. The backing membrane ensures that the health additive and adhesive are preserved prior to placement of the apparatus onto the natural fingernail.

The aspects of the present disclosure have various advantages. For instance, the protective film can provide an improved surface for decorating the individual's nail that results in long-lasting polish wear as well as a surface having actives in the film's adhesive for repairing, strengthening, and protecting the individual's nail prior to nail polish/chemicals being applied or damaged skin. Additionally, the health additives or actives are released to the nail or skin at a specific timing to improve the quality of the nail or skin.

### BRIEF DESCRIPTION OF THE DRAWINGS

Other aspects of the present disclosure will be readily appreciated, as the same becomes better understood by reference to the following detailed description when considered in connection with the accompanying drawings wherein:

FIGS. 1A and 1B are illustrations of the anatomy of a nail of an individual;

FIG. 2 is a top view of an example of a protective film for application to an individual's body in accordance with an aspect of the present disclosure;

FIG. 3 is a cross-sectional view of an example of a protective film in accordance with an aspect of the present disclosure;

FIG. 4 is an illustration of examples of various shapes of a protective film for application to an individual's body in accordance with an aspect of the present disclosure;

FIG. 5 is an illustration of an example of the application of a protective film to an individual's nail in accordance with an aspect of the present disclosure:

FIG. 6 is an illustration of an example of a protective film applied to an individual's foot in accordance with an aspect of the present disclosure;

FIG. 7 is a flowchart of a method for applying a protective film to an individual's body in accordance with an aspect of the present disclosure;

FIG. 8 is an exploded perspective view of an apparatus with a protective film for application on a fingernail in accordance with an aspect of the present disclosure; and

FIG. 9 is a bottom view of an apparatus with a protective film for application to an individual's fingernail in accordance with an aspect of the present disclosure.

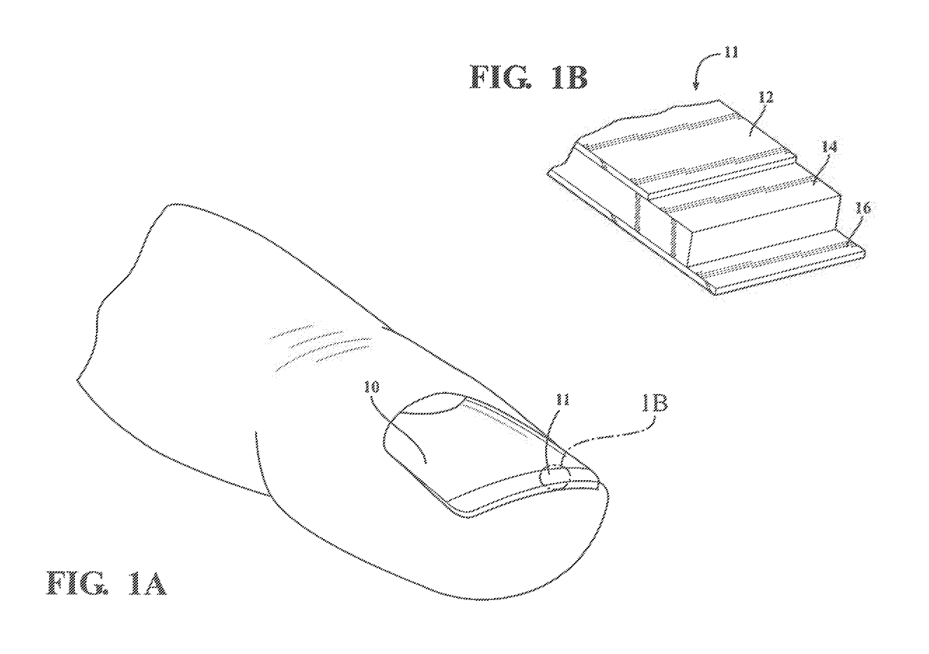

### DETAILED DESCRIPTION OF THE PRESENT DISCLOSURE

Detailed aspects of the present disclosure are provided herein; however, it is to be understood that the disclosed aspects are merely exemplary and may be embodied in various and alternative forms. It is not intended that these aspects illustrate and describe all possible forms of the disclosure. Rather, the words used in the specification are words of description rather than limitation, and it is understood that various changes may be made without departing from the spirit and scope of the disclosure. As those of ordinary skill in the art will understand, various features of the present disclosure as illustrated and described with reference to any of the figures may be combined with features illustrated in one or more other figures to produce examples of the present disclosure that are not explicitly illustrated or described. The combinations of features illustrated provide representative examples for typical applications. However, various combinations and modifications of the features consistent with the teachings of the present disclosure may be desired for particular applications or implementations. Additionally, the features and various implementing embodiments may be combined to form further examples of the disclosure.

A protective film 100 for application to a portion of an individual's body, a method of applying a protective film 100 to a portion of an individual's body, and an apparatus 130 with a protective film 100 for application to a portion of individual's body are provided. The protective film 100 and apparatus 130 with the protective film 100 are designed to be applied to an individual's nail 116 or a portion of the individual's skin 118. The protective film 100 and apparatus 130 with the protective film 100 are also designed to act as a protective barrier from damage caused by the application or removal of decorative treatment on the individual's nail 116, or one or more external elements against the portion of the individual's skin 118 such as friction generated by the individual clothes or shoes. The protective film 100 and apparatus 130 with the protective film 100 may include an active ingredient which disperses one or more health additives 114 for enhancing, improving, or repairing the condition of the individual's nail 116 or skin 118. The protective film 100 and apparatus 130 with the protective film 100 may further act as a releasing mechanism for easy removal of the decorative treatments from the nail 116. Accordingly, use of the protective film 100, the method of applying the protective film 100, or apparatus 130 with the protective film 100 provides health benefits to the individual as well as minimizes the negative effects, discussed above, caused by decorative treatments or external elements.

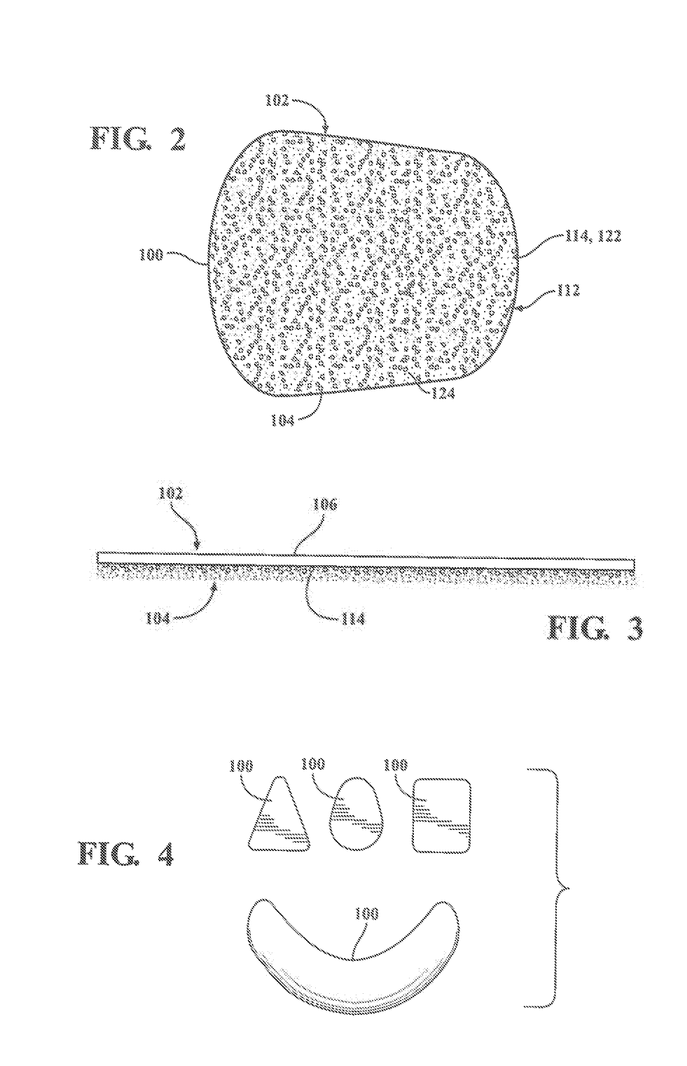

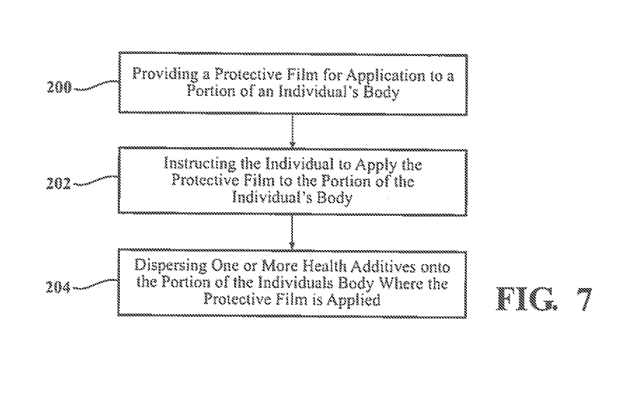

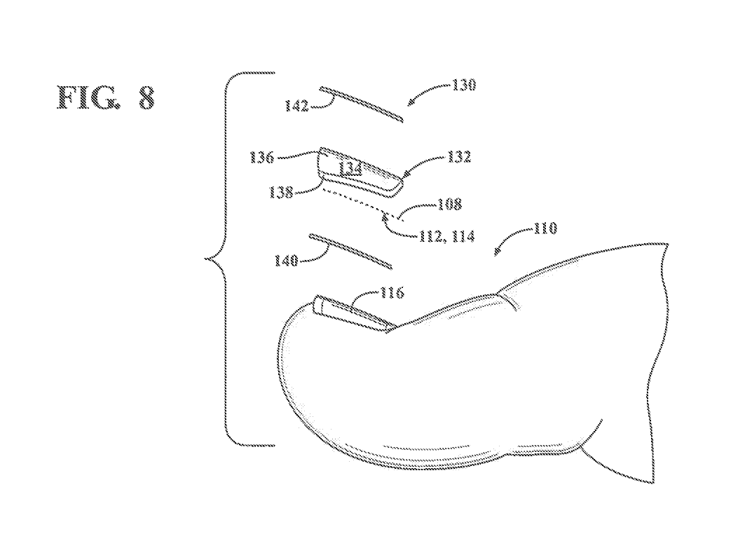

With reference to the figures, FIGS. 2-4 illustrate the protective film 100 for application on the individual's body in its broadest aspects, FIGS. 5-6 illustrate examples of different parts of the individual's body where the protective film 100 applied, FIG. 7 is a flowchart of the method for applying the protective film 100 described in FIGS. 2-4, and FIGS. 8-9 illustrate an apparatus 130 with a protective film 100 for application on the individual's body and in particular, the individual's nail 116.

According to an aspect, the protective film 100 can include a first side 102 and a second side. The first side 102 may have a smooth surface 106 for application of a decorative treatment (not shown). The second side has an adhesive 108 for securing the protective film 100 to the portion of the individual's body. The adhesive 108 can include an active ingredient 112 configured to disperse one or more health additives 114 into the portion of the individual's body 110, where the protective film 100 is applied. The portion of the individual's body 110, in which the protective film 100 is applied, may be the individual's nail 116 (fingernail or toe nail) or a portion of the individual's skin 118 such as on a callus, a blister, or a crack formed therein.

According to an aspect, the decorative treatment may include the application of a lacquer nail polish, a gel nail polish, a second adhesive and an artificial nail, or an acrylic overlay with or without an artificial nail.

According to one aspect, the active ingredient 112 with one or more health additives 114 is incorporated or infused in the adhesive 108. The active ingredient 112 and/or the health additives 112 may be time-released such that the health additives are dispersed onto the portion of the individual's body after or for a predetermined amount of time following application of the protective film to the individual's body. In another aspect, the active ingredient 112 and/or the health additives 112 may be time-released such that the health additives are dispersed continuously onto the portion of the individual's body, immediately when applied or after a predetermined amount of time following application, until the protective film is removed. The health additives 114 may include one or more vitamins, minerals, proteins, nutrients, topical medications, homeopathic treatments, repairing additives, or a combination thereof for enhancing, improving, or repairing the condition of the individual's nail 116 or skin 118. Such may include, but is not limited to, salicylic acid, urea, or carbamide. For example, where the protective film 100 is applied to a callus, the active ingredient 112 may include first health additive 114 such as salicylic acid and a second health additive. Alternatively, where the protective film 100 is applied to a callus, the active ingredient 112 may include salicylic acid, a second health additive, and a third health additive. Additionally, in a further example where the protective film 100 is applied to a callus, the adhesive 108 infused with the active ingredient 112 with one or more health additives 114 may assist in isolating the callus, such that callus becomes partially or entirely detached from the individual's skin, when the protective film 100 removed. Accordingly, the individual may see part of or their entire callus stuck to the used protective film 100.

Another aspect of the adhesive 108 may include plurality of heads 122 spaced apart from one another by a plurality of gaps 124. The plurality of beads 122 and plurality of gaps 124 may form a grid pattern. The plurality of beads 122 may entrap the active Ingredient 112 containing one or more health additives 114, which may be a time-released ingredient and may disperse one or more health additives 114 onto the portion of the individual's body 110, in which the protective film 100 is applied, after a predetermined amount of time. In an alternative aspect, the active ingredient 112 may be a non-time released ingredient and may immediately disperse one or more health additives 114 onto the portion of the individual's body 110 upon application.

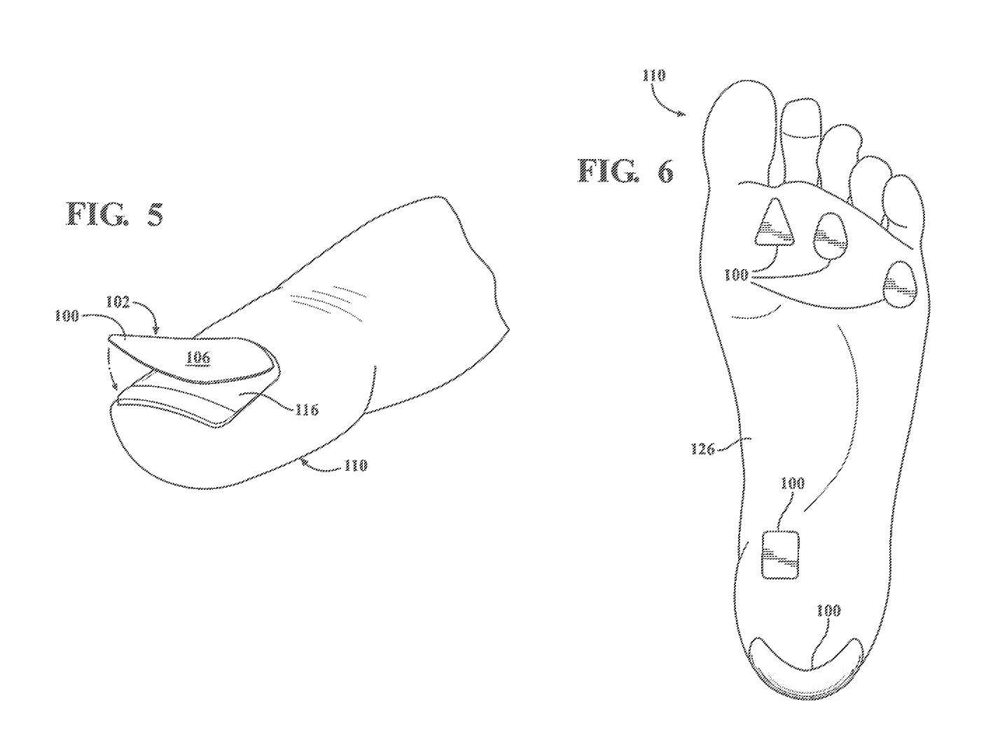

In one aspect, as shown in FIGS. 2 and 4, the protective film 100 may be pre-cut or may have a pre-determined shape to more accurately fit the portion of the individual's body. Accordingly, one or more protective film 100 be formed by a film sheet and may have a perforated edges to indicate the pre-cut or pre-determined shapes and separate the individual shapes from the film sheet. For instance, the protective film 100 may have a nail like shape for application to the individual's nail. In another example, the protective film 100 may have a rectangular, square, circular, triangular, oval, oblong, or U-like shape for application to the individual's skin. As shown in FIG. 6, one of the protective films 100 has a U-like shape for application to the individual's heel. Additionally, another protective film 100 has a triangular shape for application on the bottom of the individual's foot, where a callus may be located. Further, the protective film 100 may have the same shape as the callus on the individual's skin. In another instance, one or more shapes of the protective film 100 may be applied to an area of individual's skin to accommodate the shape of their body or to accommodate the shape of a callus area.

FIG. 5 shows the protective film 100, as discussed above and as shown in FIGS. 2-4, in the process of being applied to the individual's nail 116. Similarly, FIG. 6 shows the protective film 100, also discussed above and shown in FIGS. 2-4, on the individual's skin 118 and in particular, on the individual's foot 126. The method or process for application of the protective film 100 to the individual's body 110 and in particular, to the individual's nail 116 as shown in FIG. 5 and the individual's foot 126 as shown in FIG. 6, is described in further detail below, as depicted in FIG. 7, and may further reference FIGS. 2-6.

According to an aspect, a system and method for applying the protective film 100 to a portion of an individual body 110 is also provided. The method may include providing 200 the protective film 100 for application to the individual's body 110, instructing 202 the individual to apply the protective film 100 to the portion of an individual's body 110, and dispersing 204 one or more health additives 114 onto the portion of the individual's body 110 where the protective film 100 is applied, which may be configured to improve the condition of the portion of the individual's body 110 where the protective film 100 is applied as discussed above. In application, the protective film 100 may provide a barrier from damage associated with the application of a decorative treatment, the removal process of the decorative treatment, or one or more external elements. The method may also include instructing the individual to determine the shape of the protective film 100 based on the portion of the individual's body 110 in which the film 100 is being applied. The shape may be predetermined or pre-cut. The method may further include instructing the individual to remove a backing membrane from the second side of the protective film 100. The backing membrane may overlie and protect the adhesive 108 and health additive from exposure prior to application to the portion of the individual's body 110.

In one example, the method may enable the individual to decorate their nail 116. The system and method can be utilized in connection with either an individual's finger or toe nails 116. According to this aspect, the system can include applying a thin film 100 over an individual's nail 116, as shown in FIG. 5. The film 100 can be sized so that a single sized film 100 can be used on each nail 116. According to another aspect, the film 100 can include multiple perforations that allow the film 100 to be sized for different sized nails 116. The film 100 may be applied from the individual's cuticle to the tip of the nail 116, as also shown in FIG. 5, or vice versa. According to still another aspect, films 100 in different sizes (i.e., small, medium, or large) can also be employed (see FIGS. 4 and 6). The film 100 may be a topical film and may be constructed of a membrane material, as shown in FIGS. 2-4. It will be appreciated that the film 100 may be constructed of a variety of other suitable materials.

It is a further aspect of the present disclosure that a protective film 100 as applied to the individual's skin 118 is the same protective film or may have similar characteristic to the protective film 100 as applied to the individual's nail 116, described above. The protective film 100 and method also can be utilized in connection with the individual's skin 118 on any portion of the individual's body 110. In particular, the protective film 100 may be applied to the individual's skin 118 prior to or after any damage, such as cracks, blisters, or calluses, has occurred. The protective film 100 may be designed to act as a protective shield on the skin 118 and may also act as a repairing agent alter damage has occurred. According to an aspect, the system can include applying a thin film 100 over a portion of the individual's skin 118 on the individual's body 110. The film 100 can be sized so that a single sized filmed can be used on the portion of the individual's skin 118. The film 100 may have various sizes (small, medium, and large) and may further include multiple perforations that allow the film 100 to be sized easily for different portions of the individual's body 110, as shown in FIG. 6. The film 100 may be a topical film and may be constructed of membrane material. It will be appreciated that the film 100 may alternatively be constructed of a variety of other suitable material.

According to an aspect, the film 100 may have two sides 102, 104 and may an adhesive 108 disposed on one side for attachment to the skin 118, as shown in FIGS. 2-4. Once attached to the skin 118, the film 100 serves as a protective shield from outside elements coming into contact with the individual's skin 118. An active or an active ingredient 112 such as a health additive, repairing additive, vitamin, or medication may be included in or entrapped by the adhesive 108 that is used to secure the film 100 to the individual's skin 118. The active ingredient 112 may be a time-released active ingredient 112 that is released or dispersed into the skin 118 of the individual's body 110 after a predetermined time after the film 100 has been applied to the individual's skin 118. For Instance, the active ingredient 112 may be time-released within minutes, hours, or days after application. In an alternative aspect, the active ingredient 112 may be a non-time released active ingredient 112 that is released or dispersed into the individual's skin 118 immediately when the film 100 is applied to the individual's skin 118. The active ingredient 112 may be used to protect and give nutrients to the skin 118. Additionally, the active ingredient 112 may be used to repair the skin 118. For instance, as shown in FIG. 6, the film 100 may be placed on an individual's callus on their foot 126 to repair the individual's loot 126 and reduce the callus. It is appreciated that the protective film 100 may be utilized to repair other damage to the individual's skin 118.

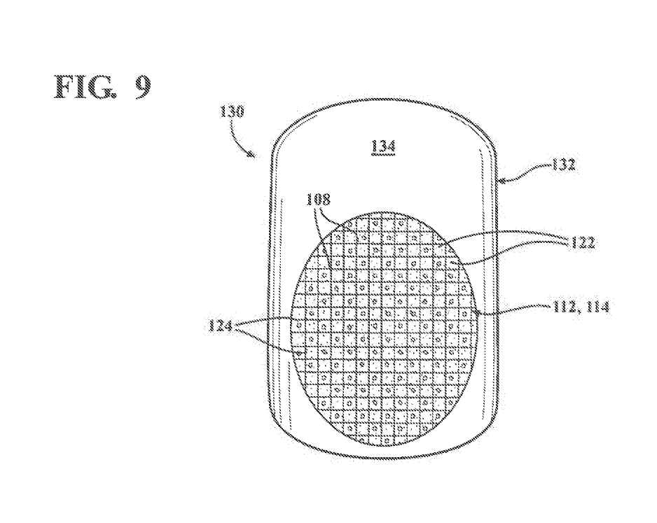

It is appreciated by one of ordinary skill in the art that the protective film 100 may be provided on its own as discussed above, or may be incorporated into another apparatus 130 as shown in FIGS. 8 and 9.

As discussed above, an apparatus 130 with a protective film 100 incorporated there is also provided. In its broadest aspect, the apparatus 130 may include an artificial nail 132 having a nail body 134 with an upper surface 136 and a lower surface 138, and an adhesive 108 disposed against the lower surface for securing the nail body 134 to a nail 116 of an individual. The adhesive 108 forms the protective film 100, which may be made of a plurality of beads 122 spaced apart from one another by a plurality of gaps 124. The plurality of beads 122 may include an active ingredient configured to disperse one or more health additives 114 onto the individual's nail 116. The protective film 100 may also form a barrier between the artificial nail 132 and the individual's nail 116 to protect the individual's nail 116 from damage caused by the application of decorative treatments on the artificial nail 132 or the removal of the artificial nail 132 from the individual's nail 116.

With reference to FIG. 8, the apparatus 130 includes an artificial nail 132 having a nail body 134 for overlying and being secured to a natural fingernail 116 of a person. In one example, the nail body 134 is made of an acrylic material; however, other materials could be utilized. When disposed on a fingernail 116, the apparatus 130 not only provides for an aesthetically pleasing covering of the fingernail 116, but also protects the fingernail 116 and delivers at least one beneficial health additive 114 to the fingernail 116.

The nail body 134 has a lower surface and an upper surface 136. The lower surface 138 is sized and configured to overlie and be secured to the fingernail 116. In one example, a portion of the lower surface engages the natural fingernail 116, and another portion extends over and away from the fingernail 116. It should be appreciated that the nail body 134 could have other shapes and sizes, e.g., the boundaries of the nail body 134 may be shaped and sized such that they are flush with the perimeter of the natural fingernail 116.

The upper surface 136 may receive one or more coatings such as nail polish 142 and finishing materials. The nail polish 142 can be used to make the apparatus 130 look like the natural fingernail 116 or it can also be used to provide other attractive and unique appearances. The finishing material can be used to protect the natural fingernail 116 and/or the nail polish 142 from damage.

As best shown in FIGS. 8 and 9, an adhesive 108 is disposed against the lower surface 138 for securing the apparatus 130 to the fingernail 116. The adhesive 108 is separated into a plurality of beads 122 that are spaced from one another by a plurality of gaps 124 in a grid pattern. In FIG. 9, the grid pattern has an outer perimeter that has a circular shape. It should be appreciated, however, that other shapes may be utilized including, but not limited to, a rectangular shape and a square shape. Additionally, it should be appreciated that the cells of the grid may have various shapes and sizes.

At least one health additive 114 is disposed against the lower surface 138 of the nail body 134 in one or more of the gaps 124 for being delivered onto the natural fingernail 116 of the person after the lower surface 138 of the nail body 134 has been applied against the fingernail 116 of the person. The health additive 114 may include various vitamins, proteins, nutrients, etc. The health additive 114 may also be homeopathic in nature. If should be appreciated that a single type of health additive 114 may be utilized, or more than one health additive 114 may be used. If more than one health additive 114 is utilized, the different types of health additives 114 may be disposed uniformly across the lower surface 138 of the nail body 134 or they may be positioned at different predetermined locations on the lower surface 138 of the body to deliver specific health additives 114 to different parts of the fingernail 116 as needed.

Based on the type of health additive 114 utilized, the health additive 114 may encourage healthy development of the natural fingernail 116 and/or inhibit the growth of bacteria or other harmful organisms between the apparatus 130 and the natural fingernail 116. Furthermore, the health additive 114 may be configured to be time-released or released immediately upon the placement of the apparatus 130 on the fingernail 116.

It should also be appreciated that the grid-shaped pattern of the adhesive 108 and health additive 114 on the lower surface 138 of the nail body 134 provides for even distribution of the adhesive 108 and health additive 114, while ensuring that the nail body 134 remains secured to the natural fingernail 116.

With reference back to FIG. 8, a backing membrane 140 removeably overlies the adhesive 108 and the health additive 114 on the lower surface 138 for covering and protecting the adhesive 108 and the health additive 114 prior to removal of the backing membrane 140 and subsequent placement of the lower surface 138 of the nail body 134 on the natural fingernail 116. Said another way, in an initial configuration, the backing membrane 140 overlies and protects the adhesive 108 and health additive 114. The backing membrane 140 may be removed from the nail body 134 into an exposed configuration in which the adhesive 108 and health additive 114 are exposed to allow the adhesive 108 and health additive 114 to be placed over the natural fingernail 116. The backing membrane 140 also ensures that that health additive 114 and adhesive 108 are preserved prior to placement of the apparatus 130 onto the natural fingernail 116.

Note that not all of the activities described above in the general description or the examples are required, that a portion of a specific activity may not be required, and that one or more further activities may be performed in addition to those described. Still further, the orders in which activities are listed are not necessarily the order in which they are performed.

The specification and illustrations of the embodiments described herein are intended to provide a general understanding of the structure of the various embodiments. The specification and illustrations are not intended to serve as an exhaustive and comprehensive description of all of the elements and features of apparatus and systems that use the structures or methods described herein. Many other embodiments may be apparent to those of skill in the art upon reviewing the disclosure. Other embodiments may be used and derived from the disclosure, such that a structural substitution, logical substitution, or another change may be made without departing from the scope of the disclosure. Accordingly, the disclosure is to be regarded as illustrative rather than restrictive.

Certain features are, for clarity, described herein in the context of separate embodiments, may also be provided in combination in a single embodiment. Conversely, various features that are, for brevity, described in the context of a single embodiment, may also be provided separately or in any sub combination. Further, reference to values stated in ranges includes each and every value within that range.

Benefits, other advantages, and solutions to problems have been described above with regard to specific embodiments. However, the benefits, advantages, solutions to problems, and any feature(s) that may cause any benefit, advantage, or solution to occur or become more pronounced are not to be construed as a critical, required, or essential feature of any or all the claims.

The above-disclosed subject matter is to be considered illustrative, and not restrictive, and the appended claims are intended to cover any and all such modifications, enhancements, and other embodiments that fall within the scope of the present invention. Thus, to the maximum extent allowed by law, the scope of the present invention is to be determined by the broadest permissible interpretation of the following claims and their equivalents, and shall not be restricted or limited by the foregoing detailed description.

Although only a few exemplary embodiments have been described in detail above, those skilled in the art will readily appreciate that many modifications are possible in the exemplary embodiments without materially departing from the novel teachings and advantages of the embodiments of the present disclosure. Accordingly, all such modifications are intended to be included within the scope of the embodiments of the present disclosure as defined in the following claims. In the claims, means-plus-function clauses are intended to cover the structures described herein as performing the recited function and not only structural equivalents, but also equivalent structures.

What is claimed is:

1. A protective film for application to a portion of an individual's body, comprising:
a first side; and
a second side having an adhesive for securing the protective film to the portion of the individual's body,
wherein the adhesive includes an active ingredient configured to disperse at least one health additive onto the portion of the individual's body where the protective film is applied, and
wherein the portion of the individual's body is at least one of the individual's nail and the individual's skin.

2. The protective film of claim 1, wherein the first side has a smooth surface for application of a decorative treatment.

3. The protective film of claim 2, wherein the decorative treatment is one of a lacquer nail polish, a gel nail polish, a second adhesive and an artificial nail, and an acrylic material.

4. The protective film of claim 1, wherein the adhesive is made of a plurality of beads spaced apart from one another by a plurality of gaps.

5. The protective film of claim 1, wherein the active ingredient is time-released and disperses the at least one health additive onto the portion of the individual's body, in which the protective film is applied, after a predetermined amount of time.

6. The protective film of claim 1, wherein the active ingredient immediately disperses the at least one health additive onto the portion of the individual's body upon application.

7. The protective film of claim 1, wherein the at least one health additive includes at least one of a topical medication and a repairing additive for repairing the condition of the portion of the individual's body to which the protective film is applied.

8. The protective film of claim 1, wherein the at least one health additive includes at least one of a vitamin, a protein, and a nutrient for strengthening the condition of the portion of the individual's body to which the protective film is applied.

9. The protective film of claim 1, the protective film is configured to protect the portion of the individual's body from damage caused by at least one of an application of a decorative treatment, a removal process of the decorative treatment, and an external element.

10. The protective film of claim 9, wherein the external element includes friction, against the individual's skin, caused by one of the individual's clothing and shoes.

11. The protective film of claim 2, wherein the protective film is a releasing mechanism for removal of the decorative treatment from the individual's nail.

12. The protective film of claim 1, wherein the individual's skin includes one of a callus, a blister, and a crack.

13. A method for applying a protective film to a portion of an individual's body, comprising the steps of
providing the protective film for application to the individual's body, the protective film having a first side and a second side with an adhesive for securing the protective film to the portion of the individual's body, wherein the adhesive includes an active ingredient configured to disperse at least one health additive onto the portion of the individual's body where the protective film is applied and wherein the portion of the individual's body is at least one of the individual's nail and the individual's skin;
instructing the individual to apply the protective film to the portion of an individual's body; and
dispersing at least one health additive onto the portion of the individual's body where the protective film is applied,
wherein the at least one health additive is configured to improve the condition of the portion of the individual's body where the protective film is applied, and
wherein the protective film provides a barrier from one of an application of a decorative treatment, a removal process of the decorative treatment, and an external element to minimize damage caused to the portion of the individual's body in which the film is applied.

14. The method of claim 13, further comprising:
instructing the individual to determine the shape of the protective film based on the portion of the individual's body in which the film is being applied.

15. The method of claim 14, wherein the protective film has a predetermined shape to fit the portion of the individual's body.

16. The method of claim 15, wherein the predetermined shape is a nail shape.

17. The method of claim 13, further comprising:
instructing the individual to remove a backing membrane from the second side of the protective film,
wherein the backing membrane overlies and protects the adhesive and health additive from exposure prior to application to the portion of the individual's body.

18. An apparatus with a protective film, comprising:
an artificial nail having a nail body with an upper surface and a lower surface;
an adhesive disposed against the lower surface for securing the nail body to a nail of an individual; and
wherein the adhesive forms the protective film and wherein the protective film is made of a plurality of beads spaced apart from by a plurality of gaps, wherein the plurality of beads include an active ingredient configured to disperse at least one health additive onto the individual's nail when the lower surface of the nail body has been applied against the individual's nail, and
wherein the protective film forms a barrier between the artificial nail and the individual's nail to protect the individual's nail from damage caused by one of an application of decorative treatments on the artificial nail and a removal process of the artificial nail from the individual's nail.

19. The apparatus of claim 18, wherein the active ingredient is a time-released ingredient and disperses the at least one health additive onto the individual's nail after a predetermined amount of time.

20. The apparatus of claim 18, wherein the at least one health additive is configured to strengthen the condition of the individual's nail.
